(19) United States
(12) Patent Application Publication
(10) Pub. No.: US 2024/0252346 A1
(43) Pub. Date: Aug. 1, 2024

(54) A male urinary incontinence device

(71) Applicant: Coloplast A/S, Humlebaek (DK)

(72) Inventors: Kim BECKER, Hilleroed (DK); Kristoffer ZEUTHEN, Copenhagen OE (DK); Rune STAGSTRUP, Broendby Strand (DK); Chaban BOUGHERARA, Frederiksberg C (DK); Malene THORUP, Bagsvaerd (DK)

(21) Appl. No.: 18/019,484

(22) PCT Filed: Jul. 12, 2021
(86) PCT No.: PCT/DK2021/050238

§ 371 Date: Feb. 3, 2023

(30) Foreign Application Priority Data
Aug. 3, 2020 (DK) PA 2020 70510

(51) Int. Cl. A61F 5/453; A61F 13/471

(52) U.S. Cl. CPC A61F 5/453; A61F 13/471

(57) ABSTRACT

A male urinary incontinence device having proximal and distal walls sealed together at the periphery to form an inner cavity. The proximal wall has a through-going inlet opening through which a penis can be entered and the inner cavity is adapted to contain at least a part of the penis. The inner cavity houses an absorber element with a volume of 25-60 cm³ (cubic centimeters), which absorber element has an absorbent core with at least 70% (w/w) super absorbent polymer (SAP).

The invention relates to a male urinary incontinence device.

### BRIEF DESCRIPTION OF THE DRAWINGS

The accompanying drawings are included to provide a further understanding of embodiments and are incorporated into and a part of this specification. The drawings illustrate embodiments and together with the description explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is an isometric exploded view of a male urinary incontinence device,

FIG. 2 is a side view and a cross-sectional view in longitudinal direction of a male urinary incontinence device,

FIG. 3 is an isometric exploded view of a male urinary incontinence device.

### DETAILED DESCRIPTION

Embodiments, and features of the various exemplary embodiments described in this application, may be combined with each other (“mixed and matched”), unless specifically noted otherwise.

In the following detailed description, reference is made to the accompanying drawings, which form a part hereof, and in which is shown by way of illustration specific embodiments in which the invention may be practiced. Because components of embodiments can be positioned in different orientations, the directional terminology is used for purposes of illustration and is in no way limiting. It is to be understood that other embodiments may be utilized and structural or logical changes may be made without departing from the scope of the present invention. Therefore, the following detailed description is not to be taken in a limiting sense, and the scope of the present invention is defined by the appended claims.

Throughout this disclosure, the words “patient” or “user” are used to address the person to wear the male urinary incontinence device. However, in some cases “user” may also relate or refer to a health care professional (HCP), such as a surgeon or continence care nurse or others. In those cases, it will either be explicitly stated, or be implicit from the context that the “user” is not the “patient” him- or herself.

In the following, whenever referring to a proximal side of a device or part of a device, the proximal side is the side closest to the user, when the device is fitted on a user and the distal side is the opposite side—the side furthest away from the user in use.

The use of the phrase “substantially” as a qualifier of certain features or effects throughout this disclosure, is intended to simply mean that any deviations are within tolerances that would normally be expected by the skilled person in the relevant field.

Many people suffer from light incontinence. Incontinence may occur in a variety of ways. Stress incontinence means that you leak urine when you sneeze, cough, laugh, lift something, change position, or do something that puts stress or strain on your bladder. Urge incontinence is an urge to urinate that is so strong that you cannot make it to the toilet in time. It also happens when your bladder squeezes when it should not. This can happen even when you have only a small amount of urine in your bladder. Overactive bladder is a kind of urge incontinence. Where the amount of leaked urine in stress incontinence may be quite small such as few drops, the amount of leaked urine in urge incontinence may be larger, such as up to 180 ml a day.

Male urinary incontinence aiding systems are known. Widely used systems known as external urinary catheters and urisheaths normally comprise a roll-on sheath or body portion for enclosing the shaft of the penis, and a tip portion that is provided with a comparatively short discharge tube, which via a tube is connected to a urine collection bag that is fastened to the leg of the user. The sheath portion is rolled-up in a number of successive windings to such an extent that adhesive on the inner surface of the sheath is accommodated in the windings. However, unrolling of a sheath correctly on a penis can be very challenging and it is essential that the sheath is fitted correctly to ensure a good sealing between the skin of the penis and the sheath. The known systems are especially intended and designed for persons suffering from medium to high urine incontinence, and the urine collection bags having a capacity of 500 ml are rather big and inconvenient to wear.

There is a need for a lightweight and discreet male urinary incontinence device that may conveniently be sealingly arranged on the penis and having a smaller capacity of about 100-300 ml and be more agreeable for the user to wear in case of lighter incontinence. There is a further need for the male urinary incontinence device to be able to very quickly and reliably absorb and contain the urine. If urine is not absorbed quickly enough, or if not all of the urine is absorbed, there is a risk that parts of the penis will be constantly exposed to liquid urine, which can lead to skin irritation, pain and skin damage, such as maceration. Incontinent users and health care professionals alike would welcome improvements in continence care devices to better meet such requirements.

The present inventors were faced with the problem of providing a device that was, at the same time, discreet, lightweight and capable of very quickly absorbing a sufficient volume of urine. The requirements for discretion and lightweight put significant limits on the size and mass of the absorbent material to be used in the device. For these reasons, the inventors had to come up with a novel absorbent element with the required characteristics.

Embodiments provide a male urinary incontinence device comprising a proximal wall and a distal wall being sealed together at the periphery to form an inner cavity, the proximal wall comprises a through-going inlet opening through which a penis can be entered, the inner cavity adapted to contain at least a part of the penis, wherein the inner cavity houses an absorber element with a volume of 25-60 cm³ (cubic centimeters), the absorber element comprising an absorbent core comprising at least 70% (w/w) super absorbent polymer (SAP).

A device with an absorber element volume of 25-60 cm³ can be made small enough to fulfil the criteria of discretion and lightweight and can, at the same time and due to the unusually high content of SAP of at least 70% (w/w) in the absorbent core, still achieve high enough absorption capacity and speed.

Embodiments provide a male urinary incontinence device comprising a proximal wall and a distal wall being sealed together at the periphery to form an inner cavity, the proximal wall comprises a through-going inlet opening through which a penis can be entered, the inner cavity adapted to contain at least a part of the penis, wherein the inner cavity houses an absorber element with a volume of 25-60 cm³ (cubic centimeters), and wherein the absorber element has an absorption capacity of at least 100 ml.

A device with an absorber volume element of 25-60 cm³ can be made small enough to fulfil the criteria of discretion and lightweight and can, at the same time and as shown herein by the present inventors, be made to achieve high enough absorption capacity. An absorption capacity of at least 100 ml ensures that the device can absorb the urine from light stress incontinence over at least an entire day and from urge incontinence for at least a one urination. This makes it possible for the user to rely on the product to absorb the urine from at least one incontinence situation after which the product can be changed if necessary.

Embodiments provide a male urinary incontinence device comprising a proximal wall and a distal wall being sealed together at the periphery to form an inner cavity, the proximal wall comprises a through-going inlet opening through which a penis can be entered, the inner cavity adapted to contain at least a part of the penis, wherein the inner cavity houses an absorber element with a volume of 25-60 cm³ (cubic centimeters), and wherein the absorber element has an absorption speed of at least 1 ml/s.

A device with an absorber element volume of 25-60 cm³ can be made small enough to fulfil the criteria of discretion and lightweight and can, at the same time and as shown herein by the present inventors, be made to achieve high enough absorption speed. An absorption speed of at least 1 ml/s ensures that the urine is absorbed quickly enough to not accumulate in the device during incontinence. This is important in order to avoid prolonged contact between urine and the penis, which can lead to skin damage.

In embodiments, the absorber element comprises super absorbent polymer (SAP), such as poly acrylic acid. SAP is generally defined as a polymer that can absorb and retain extremely large amounts of a liquid relative to its own mass. In the present context, the relevant liquid is urine and the use of SAP is particularly advantageous because of the need for something that is both small, discreet, lightweight, and highly absorbent.

In embodiments, the superabsorbent polymer comprises poly acrylic acid and/or a salt thereof, such as poly acrylic acid sodium salt, sometimes referred to as sodium polyacrylate. The superabsorbent polymer may also comprise materials selected from the group consisting of polyacrylamide copolymer, ethylene maleic anhydride copolymer, cross-linked carboxymethylcellulose, polyvinyl alcohol copolymers, cross-linked polyethylene oxide, starch grafted copolymer of polyacrylonitrile, foamed polyurethane, gelatin, and salts of any of these.

In embodiments, the absorbent core further comprises a fluff pulp material. Fluff pulp materials are often used in connection with absorbent elements and provides advantageous dry and wet strength as well as porosity, softness and water absorption. The fluff can act as a carrier for a further absorbent material, such as SAP, and can help in distributing the SAP and liquid properly in the absorbent core.

In embodiments, the absorber element comprises a proximal tissue layer and a distal tissue layer and the absorbent core between the two tissue layers. In embodiments the absorber element comprises only a single tissue layer, such as only a proximal tissue layer or only a distal tissue layer, and the absorbent core is positioned on the distal side of the proximal tissue layer or on the proximal side of the distal tissue layer. Tissue layers provide a soft material encapsulating the absorbent core of the absorber element and holding in place the absorbent material. Tissue layers can further prevent leakage of SAP from the absorbent core.

In embodiments, the absorber element is attached to the urinary incontinence device by means of welding, adhesive or mechanical fastening, such as Velcro. In embodiments, the absorber element is attached to an inside surface of the distal wall of the urinary incontinence device. Attachment to the inside surface of the distal wall means that the absorber element is located on the wall opposite the inlet opening through which the penis is inserted during use. This is advantageous because it places the absorber element in the correct position for immediately absorbing urine from the penis.

In embodiments, the absorber element is detachably attached to the urinary incontinence device. That the absorber element is detachably attached mean that it can be attached and detached without damaging the absorber element or the device. This is advantageous in that it allows for replacement of the absorber element and thus re-use of the rest of the device.

In embodiments, the absorber element has a volume of 30-50 cm³, such as 35-45 cm³, such as preferably 35-40 cm³. The absorber element needs to be small enough for the device as such to be discreet and to not feel or appear too bulky. Discretion is a key aspect of usability of male incontinence devices. The present inventors surprisingly found that it was possible to construct a discreet device with a small absorber element that was still functional in terms of intended absorption capacity and speed.

In embodiments, the absorbent core comprises 70-95% (w/w) of super absorbent polymer, such as 70-90% (w/w), such as 70-85% (w/w), such as 70-80% (w/w), such as 75-80% (w/w). In embodiments, the absorbent core comprises at least 70% (w/w) of super absorbent particles, such as at least 75% (w/w). A very high content of SAP, such as at least 70% (w/w) is one way of ensuring high enough absorption capacity and speed. Generally, a higher content of SAP also leads to higher absorption capacity and speed. Using a very high SAP content in an absorbent device can be problematic in terms of distributing the SAP homogenously and avoiding lumps in the material or leakage of SAP from the absorbent core. For these reasons, a very high content of SAP, such as above 70% (w/w) would normally not be considered for a device of the present kind. However, the present inventors surprisingly found that it was in fact possible to create a device with a very high SAP content, which provided the necessary absorption characteristics, while still getting a homogenous distribution of SAP. The present inventors found that a content of 75-80% was particularly advantageous in getting high enough absorption capacity and speed while still being able to create a homogenous absorbent core.

In embodiments, the absorber element has an absorption capacity of at least 100 ml, such as at least 125 ml, such as at least 150 ml, such as at least 175 ml, such as at least 200 ml. The absorption capacity is measured according to ISO 11948-1 (Edition 1996-11-15).

In a preferred embodiment, the absorber element as an absorption capacity of at least 180 ml. An absorption capacity of at least 180 ml ensures that the device will typically be able to absorb enough urine to last for a full day for many people suffering from urge incontinence.

In embodiments, the absorber element has an absorption speed of at least 1 ml/s, such as at least 1.1 ml/s, such as at least 1.2 ml/s, such as at least 1.25 ml/s, such as at least 1.3 ml/s. The absorption speed is measure by the following method:

Fill a flask with 125 ml 0.9% (w/w) saline solution. Fit a plastic hose with an inner diameter of 10-25 mm to the flask and adjust the flask to obtain a flow rate of 12.5+/−0.5 ml/s out of the end of the hose. Weigh an empty a glass beaker with an inner diameter of 65+/−10 mm and a minimum height of 96 mm and place the absorber element in the beaker. Adjust the absorbent element to be tilted at a 15-20 degree angle to the vertical position. Bring the end of the hose in contact with the centre of the upward tilted side of the absorber element and start the flow of saline from the flask. After 60 seconds, remove the absorber element from the beaker and weigh the beaker with the non-absorbed saline. The absorbed amount is the difference between the non-absorbed amount and the initial total volume in the flask of 125 ml (wherein 1 ml saline=1 g saline).

In embodiments, the distal wall comprises a second opening configured for allowing access to the inner cavity. When the second opening is open, it facilitates easy application of the device to the penis as both sides of the aperture are accessible and the penis can be pulled through the inlet opening and into the inner cavity. Furthermore, by opening the second opening, the user can retrieve the penis from the device, having the penis penetrating both openings, without detaching the device, and urinate naturally. Also, the second opening allows for inspection or exchange of the absorber element through the second opening.

The proximal wall and the distal wall may be welded together at the periphery. The through-going inlet opening may comprise a circular shape. The through-going inlet opening is positioned at a distance from the periphery of the proximal wall of male urinary incontinence device. The second opening may be liquid-tight and/or splash-proof closable.

The second opening may be adapted to be opened and closed several times, e.g. such as 10 times before the device may be disposed.

In embodiments, the through-going inlet opening comprises a circular shape configured to fit around the shaft of a penis.

In embodiments, the second opening is positioned opposed to the through-going inlet opening in the direction of insertion of the penis through the through-going inlet opening. This makes it easier for the user to retrieve the penis from the device, having the penis penetrating both openings, without detaching the device, and urinate naturally.

In embodiments, the distal wall comprises an upper wall portion and a lower wall portion, the upper wall portion comprising a first edge and the lower wall portion comprising a second edge juxtaposed the first edge, the first edge and the second edge defines the periphery of the second opening.

In embodiments, the first edge of the upper wall portion of the distal wall comprises a hinged flap portion adapted to cover the second opening.

In embodiments, the hinged flap portion comprising an adhesive strip layer at a proximal surface of the hinged flap portion adapted to adhere to the second edge of the lower wall portion of the distal wall. In embodiments, the second edge comprises a closure film layer adapted for forming a re-closable opening with the adhesive strip layer.

In embodiments, the hinged flap portion comprises a distal surface comprising tactile structure for providing tactile guidance for access to the second opening.

In embodiments, a distal surface of the hinged flap portion comprises a color for visually illustrating the flap for providing guidance to access the second opening.

Generally, the proximal wall and the distal wall comprises a welding zone along the periphery of the proximal wall and the distal wall. The proximal wall and the distal wall may be welded together at the periphery.

Generally, the male urinary incontinence device has a length of 160-210 mm, such as 180-190 mm and a width of 90-140 mm, such as 110-120 mm.

In embodiments, the urinary incontinence device has a total internal volume of less than 300 cm³, such as less than 250 cm³, such as less than 200 cm³, such as less than 150 cm³. A lower internal volume will make it possible to make a smaller device, which is more discrete for the user.

In embodiments, the upper wall portion comprises a radially inwardly extending sealing zone for reinforcement of the first edge adjacent along the edge of the second opening.

In embodiments, the proximal wall comprises an inlet opening rim and the through-going inlet opening formed by a flexible cuff member, the flexible cuff member may be adapted to increase in a size corresponding to the size of the inlet opening rim.

The flexible cuff member may be composed of elastic and/or flexible material, such as silicone rubber. The flexible cuff member may be attached to the inlet opening rim at a cuff member welding zone. The flexible cuff member may comprise a membrane and a tubular shaped collar portion, which defines the through-going inlet opening.

The flexible cuff member comprising the membrane and the tubular shaped collar portion are configured to allow elastic expansion of the aperture to fittingly engage with the penis.

The flexible cuff member may be flexible to an extend that the size of the through-going inlet opening may increase to a size, during mount of the device, corresponding to the size of the inlet opening of the proximal wall.

The flexible cuff member may be manipulated and stretched to similar size of the inlet opening rim allowing the user via the second opening to mount the flexible cuff member on the shaft of a penis. After mount of the device at least the tip of a penis is in the inner cavity juxtaposed in-between the proximal wall and the distal wall.

The flexible cuff member may be formed by flexible material and be very flexible and stretchable particularly in radial direction for easier mount of the male urinary incontinence device.

In embodiments, the distal wall comprises a first edge and a second edge formed by the upper and lower wall portions, which define the shaped of the second opening.

Generally, the absorber element may be attached to the lower wall portion of the distal wall by welding at an absorber support weld positioned in a distance from the second opening of 5-40 mm or less, such as 10-25 mm, 16-19 mm or less from the second edge of the lower wall portion.

The use of a male urinary incontinence device comprises the steps of; expanding the through-going inlet opening of the flexible cuff member, positioning the through-going inlet opening of the flexible cuff member around the penis by assessing the through-going inlet opening of the flexible cuff member from proximal side and through the second opening from the distal, closing the second opening by adhering the hinged flap portion the second edge of the distal wall. The use may further comprise the step of opening the hinged flap portion to allow user to urinate or inspect the absorber element and/or the inner cavity.

### DETAILED DESCRIPTION OF THE DRAWING

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

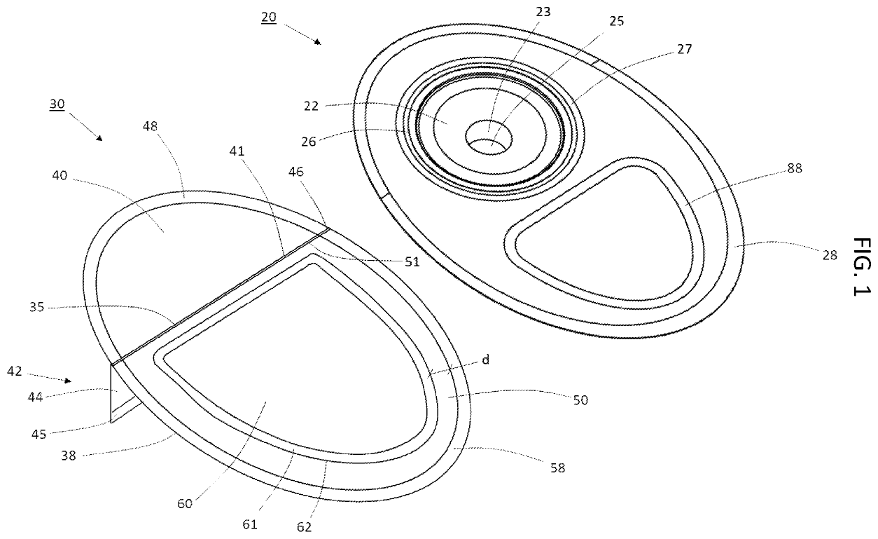

FIG. 1 illustrates the male urinary incontinence device comprising a proximal wall 20 and a distal wall 30. The periphery 28 of the proximal wall 20 and the periphery 38 of the distal wall are sealed together to form a pouch with an inner cavity 70 (as illustrated in FIG. 8). The proximal wall 20 and the distal wall 30 are welded together at the periphery.

The proximal wall 20 comprises a through-going inlet opening 25 through which a penis can be entered.

The distal wall 30 is divided into two wall portions, an upper wall portion 40 and a lower wall portion 50. The upper wall portion 40 comprising a first edge 41 and the lower wall portion 50 comprising a second edge 51 positioned juxtaposed the first edge 41. The first edge 41 and the second edge 51 of the distal wall 30 defines the periphery of the second opening 35. The second opening 35 comprises a slit-shaped form.

The upper wall portion 40 comprises a welding zone 48 and the lower wall portion 50 comprises a welding zone 58. The welding zones 48, 58 extends along the periphery 38 of the distal wall 30.

The upper wall portion 40 comprises a hinged flap portion 42. The hinged flap portion 42 overlaps the lower wall portion 50 and closes the second opening 35 formed between the upper and lower wall portions 40,50 of the distal wall 30. On the inner surface of the hinged flap portion 42 an adhesive strip layer 44 is configured to adhere to a closure film layer positioned on the second edge 51 on the lower wall portion 50 of the distal wall 30, such that the second opening is re-closeable by the adhesive strip layer being in contact with the closure film layer. The hinged flap portion 42 comprises a reinforced foam layer forming a grip portion 45.

The proximal wall 20 comprises an inlet opening rim 26. A flexible cuff member 21 is composed of elastic and/or flexible material, such as silicone. The flexible cuff member 21 is attached to the inlet opening rim 26 at a cuff member welding zone 27. The flexible cuff member 21 comprises a membrane 22 and a tubular shaped collar portion 23, which defines the through-going inlet opening 25. The proximal wall 20 and the membrane 22 extend substantially parallel in a first plane, in axial direction, and the tubular shaped collar portion 23 extends transverse to the first plane, in radial direction. The cuff member 21 may be so flexible that the through-going inlet opening 25 may obtain approx. same size as the diameter of the inlet opening rim 26.

A user may get access to the flexible cuff member 21 and the through-going inlet opening 25 through the second opening oppositely positioned the male urinary incontinence device to manipulate the size and shape of the through-going inlet opening 25 and to allow positioning of the device into position on the penis. When the tubular shaped collar portion 23 is in position, the through-going opening 25 surrounds the penis in a liquid tight manner. Thereafter the second opening may be closed by the hinged flap portion 42.

The proximal wall 20 comprises a nonwoven welding zone 88 for a comfort patch positioned at the distal surface of the proximal wall 20, to provide comfort for the user, when the device is in use.

The male urinary incontinence device comprises an absorber element 60 having a welding zone 61. The absorber element 60 is positioned adjacent the second opening 35 of the distal wall 30. The distance d between the edge 62 of the absorber element 60 and the welding zone 58 of the lower wall portion 50 is 2-4 mm or larger allowing the absorber element to increase in size when absorbing liquid, such as urine.

The absorber element 60 may comprise a heat weldable PE layer a core of absorber material and a non-women material, the PE layer and the non-women material being welded at the welding zone 61 having a width of approximately 5-6 mm and a core absorber area of approximately 6200 mm².

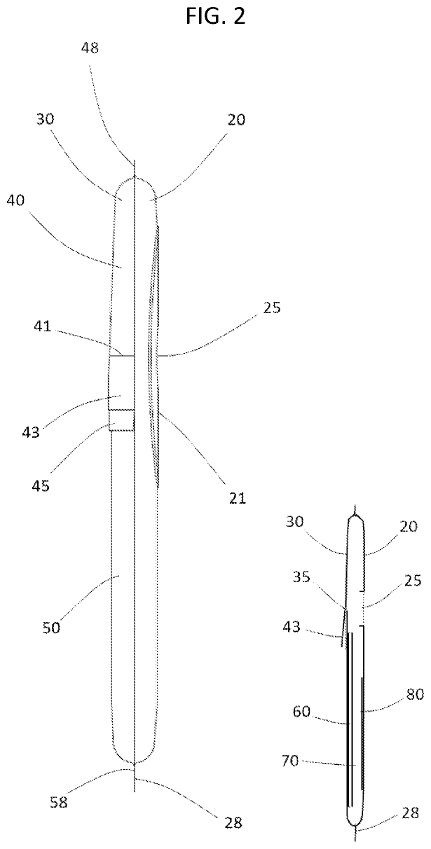

FIG. 2 illustrates a side view and a cross sectional side view of the male urinary incontinence device comprising a proximal wall 20 and distal wall 30 joint at the periphery by welding. The proximal wall 20 and the distal walls 30 encloses a volume defining the inner cavity 70. The comfort patch 80 is attached to the inner surface of the proximal wall 20 and the absorber element 60 is attached to the distal wall 30. In radial direction, the comfort patch 80 and the absorber element 60 is positioned opposed each other as well as the through-going inlet opening 25 and the second opening 35. The second opening 35 is closed off by the hinged flap portion 42, which is hingedly attached to the upper wall portion 40 at the first edge 41.

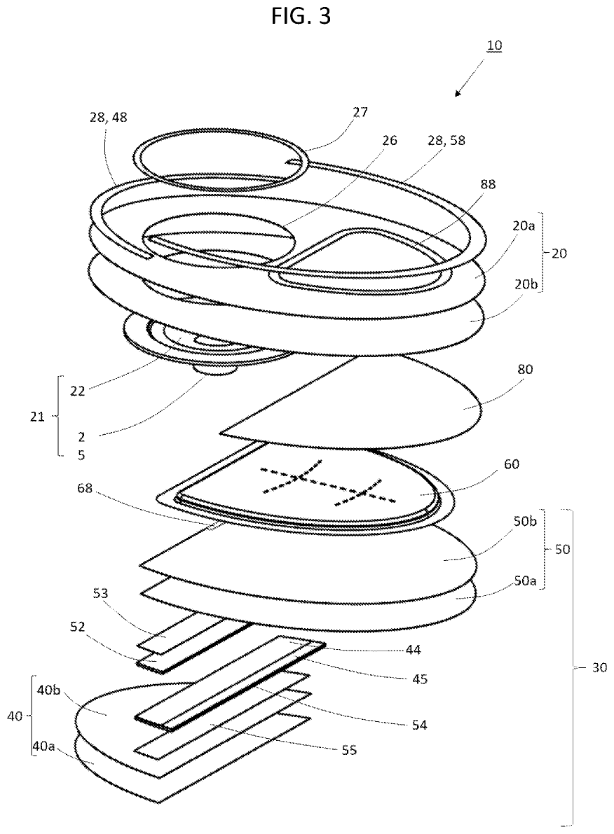

FIG. 3 illustrates an exploded isometric view of the male urinary incontinence device 10.

The device comprises the proximal wall 20 and the distal wall 30, each wall consists of a textile sheet 20a,40a,50a and a film sheet 20b,40b,50b. The male urinary incontinence device comprises sheets and layers forming a device having a compact outer shape when not in use.

The film sheets 20b,40b, 50b is provided on the inner surface of the textile sheets of the walls as to provide a liquid tight enclosure.

The different portion of the device are welded together at welding zones 27,28,48,58,88,68,53. The absorber element 60 is welded to the second edge of the lower wall portion 50.

After mount of the device at least the tip of a penis is in the inner cavity juxtaposed in-between the comfort patch attached to the proximal wall and the absorber element attached to the distal wall.

The first and second edge 41,51 of the upper wall portion 40 and the lower wall portion 50 comprises complementary closing means, in the form of an adhesive strip layer 44 and a closure film layer 52. The complementary closing means allows a user to open and close the device several times. The closure film layer 52 configured to functioning as contact surface for a hinged flap portion comprising adhesive.

The upper wall portion 40 comprises a reinforced foam layer 54 attached by welding illustrated by a welding zone 55. The foam layer 54 comprises an adhesive strip layer 44 adapted to adhere to the closure film layer 52. The protrusion of the reinforced foam layer 54 extends beyond the adhesive strip layer 44 to form the adhesive-free grip portion 45.

### EXAMPLES

An absorber element according to the present invention was created by using an EVA water-based glue to glue wood pulp tissue layers to the outside of an absorbent core comprised of fluff pulp mixed with super absorbent polymer in the form of sodium polyacrylate.

The components of the absorbent core were:

| | |
|---|---|
| Fluff pulp in absorbent core: | 1.75 g (23% (w/w)) Southern Pine |
| | cellulose fluff from GP Cellulose |
| SAP in absorbent core: | 6.0 g (77% (w/w)) of sodium polyacrylate |
| | SAP from Formosa Industries Co. Ltd. |

The absorber element had a roughly rectangular shape with an area of 62 cm² and had a depth of 0.6 cm. The volume of the absorber element was 37.2 cm³. The absorption capacity and speed of the absorber element were tested with the following results.

TABLE 1
Absorption speed

| | Volume | Time | Non-absorbed | Absorbed | Absorption speed |
|---|---|---|---|---|---|
| Sample | (ml) | (sec) | (g) | (g) | (ml/s) |
| 1.1 | 125 | 60 | 44.32 | 80.68 | 1.34 |
| 1.2 | 125 | 60 | 43.93 | 81.07 | 1.35 |
| 1.3 | 125 | 60 | 40.52 | 84.48 | 1.41 |
| 1.4 | 125 | 60 | 38.11 | 86.89 | 1.45 |
| Average | | | 41.72 | 83.28 | 1.39 |

The results for the absorber element show an average absorption speed (measured as described herein above) of 1.39 ml/s. This is high enough to achieve sufficient absorption of urine in a male urinary incontinence device as defined herein.

For the same construction of an absorber element the absorption capacity was measured according to ISO 11948-1 (Edition 1996-11-15). The results were as follows.

TABLE 2
Absorption capacity for 6 g SAP

| | Dry weight | Wet weight | Total absorption |
|---|---|---|---|
| Sample | (g) | (g) | (ml) |
| 2.1 | 8.58 | 208.71 | 200.13 |
| 2.2 | 8.86 | 208.26 | 199.40 |
| 2.3 | 8.93 | 211.04 | 202.11 |
| 2.4 | 8.80 | 203.51 | 194.71 |
| 2.5 | 9.36 | 219.20 | 209.84 |
| Average | | | 201.24 |

As can be seen, the absorption capacity is around 200 ml, which as explained above is sufficient for a male urinary incontinence device of the present type.

1-15. (canceled)

16. A male urinary incontinence device comprising a proximal wall and a distal wall being sealed together at the periphery to form an inner cavity, the proximal wall comprises a through-going inlet opening through which a penis can be entered, the inner cavity adapted to contain at least a part of the penis, wherein the inner cavity houses an absorber element with a volume of 35-45 cm³ (cubic centimeters), the absorber element comprising an absorbent core comprising 75-85% (w/w) super absorbent polymer (SAP) in the form of poly acrylic acid particles, and wherein the absorber element has an absorption speed of at least 1.25 ml/s.

17. The male urinary incontinence device according to claim 16, wherein the absorbent core further comprises a fluff pulp material.

18. The male urinary incontinence device according to claim 16, wherein the absorber element comprises a proximal tissue layer and a distal tissue layer and the absorbent core between the two tissue layers.

19. The male urinary incontinence device according to claim 16, wherein the absorber element is attached to the urinary incontinence device by means of welding, adhesive or mechanical fastening.

20. The male urinary incontinence device according to claim 16, wherein the absorber element is attached to an inside surface of the distal wall of the urinary incontinence device.

21. The male urinary incontinence device according to claim 16, wherein the absorber element is detachably attached to the urinary incontinence device.

22. The male urinary incontinence device according to claim 16, wherein the absorber element has a volume of 35-40 cm³.

23. The male urinary incontinence device according to claim 16, wherein the absorber element has an absorption capacity of at least 100 ml

24. The male urinary incontinence device according to claim 16, wherein the through-going inlet opening comprises a circular shape.

25. The male urinary incontinence device according to claim 16, wherein the proximal wall comprises an inlet opening rim and the through-going inlet opening is formed by a flexible cuff member, the flexible cuff member allowing the inlet opening to increase in size.

26. The male urinary incontinence device according to claim 16, wherein the distal wall comprises a second opening configured for allowing access to the inner cavity.

27. The male urinary incontinence device according to claim 16, wherein the proximal wall and distal wall material is a polyethylene.

28. The male urinary incontinence device according to claim 16, wherein the absorber element has an absorption capacity of at least 125 ml.

29. The male urinary incontinence device according to claim 16, wherein the absorber element has an absorption capacity of at least 150 ml.

30. The male urinary incontinence device according to claim 16, wherein the absorber element has an absorption capacity of at least 175 ml.

31. The male urinary incontinence device according to claim 16, wherein the absorber element has an absorption capacity of at least 200 ml.

32. A male urinary incontinence device comprising a proximal wall and a distal wall being sealed together at the periphery to form an inner cavity, the proximal wall comprises a through-going inlet opening through which a penis can be entered, the inner cavity adapted to contain at least a part of the penis, wherein the inner cavity houses an absorber element with a volume of 35-45 cm³ (cubic centimeters), the absorber element comprising an absorbent core comprising 75-85% (w/w) super absorbent polymer (SAP) in the form of poly acrylic acid particles, and wherein the absorber element has an absorption capacity of at least 125 ml.

33. The male urinary incontinence device according to claim 32, wherein the absorber element has an absorption capacity of at least 175 ml.

34. The male urinary incontinence device according to claim 32, wherein the absorber element has an absorption capacity of at least 200 ml.

35. The male urinary incontinence device according to claim 32, wherein the proximal wall and distal wall material is a polyethylene and the absorber element has an absorption capacity of at least 200 ml.
